(19) United States
(12) Patent Application Publication
(10) Pub. No.: US 2009/0291485 A1
(43) Pub. Date: Nov. 26, 2009

(54) APPARATUS AND METHOD FOR OPTIMIZING PHOTOSYNTHETIC GROWTH IN A PHOTO BIOREACTOR

(76) Inventors: Steven Shigematsu, Santa Rosa, CA (US); Nicholas Eckelberry, Los Angeles, CA (US)

Correspondence Address: WESLEY B. AMES, 7031 LOS VIENTOS SERENOS, ESCONDIDO, CA, 92029, US

(21) Appl. No.: 12/126,842

(22) Filed: May 23, 2008

(51) Int. Cl. C12N 1/12; C12M 1/00; F21V 21/00; C12M 1/36

(52) U.S. Cl. 435/257.1; 435/292.1; 435/286.1; 362/249.14

(57) ABSTRACT

A system and methods for enhancing mass production of algae, diatoms or other photosynthetic organism is described. The system and methods are useful in applications such as energy production, fuels, foods, pharmaceuticals, plastics, and CO₂ fixation. The system and methods involve the use of a submersed rotating pole(s) on which lights are branched out at strategic intervals in order to appropriately increase contact between photosynthetic water-based organisms and light. Rotation can timed so as to disperse light and/or nutrients when the organism is in a receiving mode. A process flow system is also described which can be scaled for the mass production of photosynthetic based organism. This process system makes use of centralized automatable controls and linear or parallel growth tanks. The system can be used for continuous or batch methods of production of bio-mass whose values such as lipids or carbohydrate can be extracted for the production of fuels or other byproducts.

### RELATED APPLICATIONS

Not applicable.

### FIELD OF THE INVENTION

The present invention relates to apparatus and methods for increasing photosynthetic growth in a photobioreactor system.

### BACKGROUND OF THE INVENTION

The following discussion is provided solely to assist the understanding of the reader, and does not constitute an admission that any of the information discussed or references cited constitute prior art to the present invention.

Natural light consists of a continuous spectrum of wavelengths, and a portion of this spectrum, ultraviolet or UV rays, is recognized as harmful to algal growth while other, relatively narrow bands of the light spectrum are recognized as critically important for optimum growth. In photosynthesis, light must first be absorbed in order to produce conversion of CO2 to carbohydrate. Continuous full frequency light is wasteful because algae cannot use light while it is absorbing and fixing carbon as part of its metabolic process, and because a significant fraction of the light energy is wasted because it is not utilized in the photosynthetic process. Therefore, extraneous light frequencies may beneficially be reduced or eliminated. It has also been determined that the frequencies of light for most effective photosynthesis and growth vary for different species being grown.

For efficient algal growth, light must therefore be distributed at the right frequencies and only for the time required for the photosynthesis process to occur. This time frame is roughly 1 second of light exposure for the initial reactions to occur, with 6 seconds for the conversion process of fixing CO2 into a carbohydrate. Previously described methods and apparatus for culturing algae in mass quantities generally fail to properly provide appropriate light exposures for optimal algal growth.

In outdoor raceways, for example, UV exposure from the full spectrum natural light results in algae at the surface of the water oxidizing, algae in secondary levels receiving some light in a limited fashion, and algae in tertiary layers receiving little light and therefore dying degrading, creating a anaerobic bio-mass that affects the overall growth of the raceway. Attempts to solve this problem led to the creation of shallow ponds or raceways. However, such shallow water approaches engender the problem of high evaporation and saline deposits, which also reduces the efficacy of continuous outdoor growth. Weather, diurnal cycles and invasion by opportunistic species further aggravate the difficulties of mass algae culturing in outdoor settings.

It has therefore been determined that growing algae in a photo-bio-reactor (PBR) would be the preferred method of mass culturing algae provided energy costs are dramatically reduced and a compact infrastructure is designed that addressed the concerns of light and nutrient delivery while providing for high density growth of fresh and sea-water organisms. For most applications, the system must also be scalable to suit the escalating fuel and electricity demands of the planet.

In current PBRs, the costs in lighting and energy requirements have made prior solutions impractical for all but the culturing of organisms used in high value products in pharmaceuticals or neutraceuticals.

In early work by William Oswald described in U.S. Pat. No. 3,520,081, a rotating tank that enhances contact between algae and light to accelerate algae growth was used. Oswald points to the inherent problem of mass culturing of algae as providing a suitable environment for optimum growth since algae growth as a mass feed stock is limited by two factors: close proximity to light and nutrients. Additionally algae species, in particular high growth genus with valuable attributes such as lipid or carbohydrate content (TAGs), such as Nanochloropsis, chlorella and others, all require specific light frequencies and nutrients for accelerated growth. And while the use of rotating tanks has some benefits, the impracticality of scale becomes apparent when discussing very large scale systems, e.g., multi million gallon systems.

Robinson et al., U.S. Pat. No. 5,137,828, suggests that a central core of light within a tube would enhance production by bringing the algae mass in contact with light and nutrients. This method has the benefits of lowering the land mass required for mass algae production and filtering out unwanted UV light frequencies by the strategic uses of reflective surfaces. Distinct problems with this method were fouling of the tube's surface by organic matter and the small industrial scale.

Yang et al., U.S. Pat. No. 5,614,378, addresses the problem of fouling with a cleaning system incorporated within a network of optical fibers in a life support system that generates oxygen. Scaling of this system to mass culturing is impractical and indeed not the intent of this PBR, as it is designed for life support in space.

Muhs et al., U.S. Pat. No. 6,603,069, describes a method of capturing light from a solar collector which feeds light at the correct frequency through a network of fiber-optics into a bio-reactor.

Hirabayashi et al. U.S. Pat. No. 6,579,714 describes an algae culture apparatus and method utilizing a growth apparatus having spaced apart inner and outer walls which are dome-shaped, conical, or cylindrical. Light can pass through the walls into the space between where the algae are cultured.

Yogev et al., U.S. Pat. No. 5,958,761 describes a “bioreactor for improved productivity of photosynthetic algae [which] includes a tubular housing surrounding a tubular envelope located therein. The housing and envelope define a space there between to be filled with fluid. The housing and envelope are made of at least a translucent material and have inlet and outlet ports providing access to the space and the interior of the envelope. A mixer for mixing algae media is disposed inside the envelope. There is also provided a bioreactive system, wherein the envelope contains a fluid of selective refractive index and wherein, for a given geometrical relationship between the housing and the envelope, the radiation concentration power is controlled by modifying the refractive index of the fluid.”

Raymond, U.S. Pat. No. 4,253,271 describes an apparatus and process for the culture of algae in a liquid medium in which the medium circulates through an open trough and is exposed to an atmosphere which is temperature regulated. The nutrient content of the liquid medium is regulated to control the chemical composition growth and reproduction characteristics of the cultured algae. Before it is allowed to strike the medium, sunlight is passed through a filter to remove wavelengths which are not photosynthetically active. Heat energy can be recovered from the filter.

In another study, the use of rotating annular reactors with lights placed within was examined. (Zittelli, Rodolfi, and Tredici, 2003, Mass cultivation of Nannochloropsis sp. In annular reactors, J Appl Phycology 15:107-114.) The annular reactor consisted of two 2-m-high Plexiglas cylinders of different diameter placed vertically one inside the other so as to form an annular culture chamber. Artificial illumination was supplied by lamps placed inside the inner cylinder.

### SUMMARY OF THE INVENTION

This invention provides a solution to vexing problems in culturing of photosynthetic microorganisms, especially algae. In particular, prior photo bioreactors have suffered from a number of difficulties which have inhibited broad application of the reactors for bulk applications, including high energy utilization, fouling of light emitting surfaces, and diurnal growth cycles. This invention addresses those problems with a system that provides efficient light utilization with comparatively low energy costs. One feature of this approach is to provide the light at closely spaced intervals within a photobioreactor so that light is provided throughout the photobioreactor rather than just at the surface and/or at culture medium/photobioreactor wall interfaces.

Thus, a first aspect of the invention concerns a culture system for photosynthetic microorganisms which includes at least one culture tank, at least one light array positioned with the tank, where the light array includes a plurality of light emitting projections (e.g., light wands or bars) positioned such that most and preferably essentially all medium passing between adjacent light emitting projections will receive photosynthetically effective illumination, and a drive system for moving the light array within the tank and/or a fluid impeller, e.g., a mixer and/or a pump. The fluid impeller causes growth medium in the tank to pass through the light array.

An advantageous embodiment of the system includes at least one rotatable light array positioned within the tank, where the light array includes an axle, a rotational drive connection linked to the axle, a plurality of light emitting projections (e.g., light wands or bars) extending outward from the axle and positioned such that most and preferably essentially all culture media passing between adjacent light emitting projections will receive photosynthetically effective illumination, and a rotational drive linked to and providing power through the rotation drive connection to rotate the rotatable light array.

In certain embodiments of the system incorporating a rotatable light array, the array includes a plurality of flat arrays distributed along the axle, e.g., at least 3, 4, 5, 7, 10, 15, 20, 30, 40, or 50 such flat arrays or is in a range defined by taking any two of the specified numbers of flat arrays as inclusive endpoints; the flat arrays are spaced at distances of 0.25 to 10 cm, 0.5 to 10 cm, 0.5 to 7 cm, 0.5 to 5 cm, 0.5 to 4 cm, 0.5 to 3 cm, 0.5 to 2 cm, 1 to 10 cm, 1 to 7 cm, 1 to 5 cm, 1 to 4, 1 to 3 cm, 2 to 10 cm, 2 to 7 cm, 2 to 5 cm, 2 to 4 cm, or 2 to 3 cm along the axis of the axle, with the distance referring to either the center-to-center distance or to the separation between successive wands; the flat array includes at least 2, 3, 4, 5, 7, 10, 15, or 20 wands or is in a range defined by taking any two of the specified values as inclusive endpoints; the rotatable light array rotates at 0.2 to 20, 0.2 to 15, 0.2 to 10, 0.2 to 5, 0.2 to 3, 0.2 to 2, 0.5 to 20, 0.5 to 10, 0.5 to 5, 0.5 to 4, 0.5 to 3, 0.5 to 2, 1 to 10, 1 to 5, 1 to 4, 1 to 3, or 1 to 2 rpm; the axle is positioned essentially vertically in the tank; the axle is positioned essentially horizontally in the tank.

In some embodiments, a light array include essentially parallel light wands, e.g., in an essentially planar array; a planar array includes at least 5, 10, 15, 20, 25, 30, 35, 40, 50, 70, or 100 light wands, or is in a range defined by taking any two of the specified values as inclusive endpoints.

Also in particular embodiments, the light array(s) is moved in the culture such that illumination is provided to a particular volume within the tank repetitively with average repeats of 1 to 20, 1 to 15, 1 to 10, 3 to 20, 3 to 15, 3 to 10, 4 to 10, 4 to 8, or 5 to 7 seconds; the wavelengths of light emitted from the light wands is selected to provide effective photosynthesis while reducing power consumption, e.g., having a luminance peak within the range of 400 and 720 nm and lower luminance outside that band, or with luminance peaks within one or both of the bands of 400 to 510 nm and 600 to 720 nm with lower luminance outside those bands; the peak of luminance emitted in the ultraviolet radiation in the range of 15 to 400 nm (or ranges of 15 to 350, 50 to 400, 50 to 350, 100 to 400, or 100 to 350 nm) is no more than 50, 40, 30, 20, 10, or 5% of the highest luminance peak in the range of 400 to 720 nm.

In certain embodiments, the system also includes at least one channel (and may include at least one reservoir) for distributing nutrients to culture media in the tank, e.g., CO2 and/or nitrogen (for example as nitrates).

In some systems, a plurality of light arrays are included in a single tank, e.g., at least 2, 3, 4, 5, 7, 10, 15, or 20 such arrays, or a number of arrays in a range defined by taking any two of the specified values as inclusive endpoints.

Advantageous embodiments include a light controller which controls at least one of the parameters of light intensity, light delivery periodicity, light duration, and light wavelength for light emitted from the light wands; the system includes a controller which controls delivery of at least one nutrient to the culture (e.g., CO2 and/or nitrogen such as in the form of nitrates); the system also includes at least one culture medium sensor, e.g., a sensor(s) producing signals corresponding to pH and/or oxidation reduction potential (ORP) and/or turbidity, preferably a controller receives signals from the sensor and controls light emitted from said light wands at least in part as a function of said signals and/or controls delivery of at least one nutrient (e.g., CO2 and/or nitrogen) to the culture tank.

A related aspect concerns a light distribution array in which a plurality of light bars or wands are positioned such that most, and preferably substantially all, culture medium passing between successive light bars is within one growth plane of a light bar.

In particular embodiments, the light distribution array is as described for the preceding aspect.

In certain embodiments, the array includes an axle (preferably rotatable) and a plurality of light emitting wands extending from and distributed along said axle. The wands may for example, be spaced at distances of 0.25 to 10 cm, 0.5 to 10 cm, 0.5 to 7 cm, 0.5 to 5 cm, 0.5 to 4 cm, 0.5 to 3 cm, 0.5 to 2 cm, 1 to 10 cm, 1 to 7 cm, 1 to 5 cm, 1 to 4, 1 to 3 cm, 2 to 10 cm, 2 to 7 cm, 2 to 5 cm, 2 to 4 cm, or 2 to 3 cm along the axis of the axle, with the distance referring to either the center-to-center distance or to the separation between successive wands. In embodiments of other configurations, e.g., a planar array, successive light wands can be distributed at distances or separations as just specified for the axle-type array.

In certain embodiments (e.g., for an axle-type array), the light distribution array of includes a plurality of flat arrays of light emitting wands; the light emitting wands in an array are distributed over a distance of at least 0.2, 0.3, 0.4, 0.5, 0.7, 1.0, 1.5, 2.0 meters, or more, e.g., along an axle in an axle-type array or in a plane in a planar array; the light emitting wands include at least one light source which is an LED, a cold cathode fluorescent light (CCFL), or an external electrode fluorescent light (EEFL).

Another aspect of the invention concerns a system for carbon fixation and/or product recovery which includes a culture system (e.g., as specified for the first aspect above or otherwise described herein for the invention), a process controller which monitors at least one culture parameter indicative of photosynthesis or growth (e.g., pH, ORP, and/or turbidity) and regulates light (e.g., light duration, light delivery period, light intensity, and/or light wavelength) and/or nutrient delivery (e.g., CO2 and/or nitrogen such as in the form of nitrates) to the culture.

In certain embodiments, the system includes a culture tank, at least one rotatable light array positioned within the tank, where the light array includes an axle, a plurality of light emitting projections extending outward from the axle and distributed along the axle, and positioned such that essentially all culture medium passing between adjacent light emitting projections will receive photosynthetically effective illumination, and a rotational drive connection. The system also includes a rotational drive which is linked to and provides power through the rotation drive connection to rotate the rotatable light array.

In certain embodiments, the system also includes an oil extractor, e.g., extracting lipids from the cultured microorganisms, and/or a biomass digester which receives biomass from the culture tank. In some embodiments, CO2 generated from a biomass digester is used as a nutrient in a culture tank.

The system can also include an electrical generator powered by at least one product of the system, e.g., an oil-based fuel such as biodiesel, biomass, or methane.

A related aspect of the invention concerns a method for growing photosynthesizing microorganisms, including exposing photosynthetic microorganisms (e.g., algae, diatoms, or photosynthetic bacteria) in a growth medium in a photobioreactor to photosynthetically effective light from a light array, where the light array includes a plurality of light wands spaced such that substantially all of the growth medium between successive light wands in the light array receive photosynthetically effective illumination.

In particular embodiments the light array is as specified for an aspect above or otherwise described herein for the invention.

Also in particular embodiments, the microorganisms are grown in a system as specified for an aspect above or otherwise described herein.

As used herein, the term “growth plane” refers to a volume of water irradiated by light in which photosynthetic growth of suspended photosynthetic microorganisms will effectively occur (assuming other growth requirements for such growth are also satisfied). For example, for solar irradiation of algal cultures in ponds, the growth plane commonly extends only about one to a few centimeters down from the surface. In the present apparatus having moving light arrays, a growth plane is defined by the distance light emitted from a light wand will be photosynthetically effective. Thus, successive light wands may be placed two growth planes apart and the entire space between will receive photosynthetically effective illumination.

In reference to light present in a photosynthetic culture medium, the term “photosynthetically effective” means the intensity of photosynthetically active radiation (PAR) is sufficient for the organism being cultured to perform photosynthesis effectively such that there is net fixation of CO₂.

Additional embodiments will be apparent from the Detailed Description and from the claims.

### BRIEF DESCRIPTION OF THE DRAWINGS

FIG. 1 shows a schematic cross-section of a portion of an exemplary light rod for the present invention.

FIG. 2 shows a schematic top view of an exemplary flat light array.

FIG. 3 shows a schematic view of an integrated growth and recovery system utilizing rotating light arrays in growth chambers.

### DETAILED DESCRIPTION OF THE PREFERRED EMBODIMENTS

### General Discussion

The use of cultivation systems for the growth of algae to be used as feed stock, e.g., for fuels, food, and plastics, as well as for other purposes, has come under intense scrutiny in the past few years. While many devices and schemes have been proposed for both outdoor (e.g., raceway) and indoor (photobioreactor) cultivation (see, e.g., Background), addressing proper lighting and nutrient dispersal had not been adequately resolved in order to attain high, consistent growth of the cultured organisms with sufficiently low costs.

The present invention directly addresses the issues of proper lighting and nutrient dispersal in a photobioreactor system, by utilizing a light array (or set of light arrays), usually a rotating light array, which can also be configured to disperse nutrients in the growth medium.

Even though exemplary uses of the present light arrays are in production of algal lipids, e.g., for production of biodiesel, and in overall biomass production, they can also be used in other applications, e.g., in waste treatment. For example, the light arrays can be incorporated in systems such as waste water treatment aeration tanks or outdoor raceway ponds. The advantage is delivery of a consistent light source at the right frequency to the whole of a bio-mass regardless of natural light vagaries.

### Light Arrays and Array Configurations

The present systems incorporate light arrays which are configured such that photosynthetically effective illumination is provided to substantially all of the culture medium volume within the volume described by the light array. One or more such light arrays may be used in each culture tank.

An example of such a light array has a rotating axle on which a plurality of light wands or light bars are attached, distributed along the axle. (The description herein emphasizes rotating light arrays, but also applies to other light array configurations except in particular instances where it is clearly only applicable to the rotating configurations.) In many cases, light wands are also distributed around the axle much like branches of a tree, e.g., forming a flat array of light wands. The light wands are distributed along the axle such that there are relatively narrow gaps between the paths described by successive light wands. For example, when the light wands are arranged in successive flat arrays, there is a plurality of such flat arrays distributed along the axle with relatively small gaps between successive flat arrays. The light wands can alternatively be distributed in various other ways, such as spiral or helix positioning, alternate offset positioning, and others. Highly advantageously, the spacing between successive light wand paths is selected such that photosynthetically effective light is provided across the entire space between the successive light wand paths, taking into consideration the intensity of the lights and the expected turbidity of the medium as the culture grows. In many cases, the spacing of the light wands will be in a range of about 2 to 10 cm, more commonly about 2 to 5 cm.

While rotating light arrays are advantageous, other configurations and other movement patterns can also be used which similarly provide the close spacing between successive light wands. In one illustrative configuration, a light array is arranged as a set of parallel light bars (preferably close-set light bars). The parallel light bars can be linked substantially in a plane, forming a planar array. Such a planar array can be moved generally linearly within a culture tank to provide light to photosynthesizing microorganisms. The linear movement would often be in a back-and-forth manner. That is, the movement would usually be in a direction substantially orthogonal to the plane of the array. Thus, in a rectangular tank, for example, a vertical array of parallel lights may be moved along an axis of the tank in a back-and-forth pattern or in a cyclic pattern, e.g., in which the array moves linearly along the upper portion of the tank, then moves down and returns moving linearly along the lower portion of the tank. A plurality of such planar arrays can be utilized in a single tank, e.g., such that a particular planar array traverses only a portion of the tank, e.g., thereby increasing the illumination frequency.

Similarly, an array the same or similar to those described for the rotating light arrays can be used with linear movement. For example, a light array with a plurality of flat arrays of light wands attached to and extending from a central axle can be moved along the axis of the axle in a reciprocal manner. Once again, a plurality of such light arrays may be used in a particular culture tank.

Other such configurations of light emitting elements arranged and/or moved such that most or substantially all of the photosynthesizing microorganisms in a culture tank will be in close proximity to the emitted light may also be used.

For any of the light wand configurations, it is highly beneficial if substantially all of the culture medium that passes between successive light wands is exposed to light (desirably to photosynthetically effective light) from the light wands. Such exposure is generally a function of the light source parameters along with the light wand distribution and medium turbidity. The light wand parameters include the selection, placement, and orientation of the light sources on the light wands, be they LED, fluorescent, cold cathode fluorescent, or other. As indicated above, the light wands are located such that the separation between successive light wand rotational paths is such that a gap is defined by movement (e.g., rotation) of the array. This gap is preferably sized and configured such that, in combination with the light source parameters, substantially no area within that gap is dark. Thus, the light distributing axle has the advantage of placing light and/or nutrients closer to the biomass.

The sizing of the gap can also be selected to create a flow-through for the suspended bio-mass, effectively generating, through Bernoulli's principles, natural high and low pressure zones that enhance flow-through and/or promotes the creation of minor eddies which in turn promote cell growth.

The present light arrays are highly adaptable to a wide variety of different culture system requirements. For example, when used in a culture system, the light array may be fixed in place, such that it is not readily and rapidly repositionable or removable, but alternatively and advantageously the light array can be configured such that it can be readily removed, replaced, and/or repositioned within a culture tank. An advantage of such mobility is that the light source (i.e., the light array) can thus be moved in and out of the culture tanks rather than moving the mass of organism culture medium, and also can be conveniently adapted to various types of cultures and culture conditions. In addition, the light array can be positioned vertically or horizontally (or in any other desired orientation) in the culture tank.

Such arrays can be used in various applications, including in culture which maximizes biomass and/or numbers of organisms (which can be referred to as biomass growth), as well as in other applications such as waste treatment (which can be referred to as process growth). The light arrays can also be used in culture tanks for which the emphasis is on increasing the amount or fraction of a desired product or products, e.g., lipids (such culturing can be referred to as product growth).

In some cases, a system may include separate culture tanks or different process stages for increasing cell number and for increasing the level of a particular product. The culture conditions may differ for those different purposes, including provision of different levels and/or frequencies of light. Thus, for example, when used in a tank for biomass growth, the light array may, for example, deliver a higher density light delivery system as compared to a product growth tank. Thus, in a product growth tank, an array with fewer and/or less intense lights and/or lights of different frequencies can be placed in growth tanks. In some cases, such product grow tanks would have the light arrays placed essentially horizontally. For example, these product growth tanks can used to increase TAG, lipid or carbohydrate values in organisms and, in many cases, would require less light and/or the light can be programmed to enhance production of the desired products, e.g., in approximate diurnal cycles. In some configurations, the lights in growth tanks can be under the control of an electronic light controller(s) that increases or decreases illumination frequencies and/or changes the light wavelengths, e.g., to promote lipid production or production or other valuable product(s).

Even though the use of movement of light arrays will typically be more efficient, it is also possible to rely on fluid movement rather than light array movement to bring cells and light source into close proximity. For example, one or more light arrays may be provided in a tank, and culture medium containing cells pumped gently through the tank or the culture medium may be gently mixed such that cells will cycle past the light sources. Similar to the situation with the rotating or otherwise moving light arrays, medium with cells will pass in close proximity to the light sources and therefore will receive photosynthetically effective illumination.

The effectiveness of the present light array design is supported by a study on correlations between certain illumination parameters and growth rates for particular algal species. Initially it was shown that there is poor correlation between the incident light intensities, the average light intensities and the light energy supplied per unit photobioreactor volume (Eₜ/V and linear growth rates of Chlorella pyrenoidosa in cuboidal photobioreactors of various sizes. It was also found that at a given Eₜ/V, the linear growth rates decreased with increase in the photobioreactor depth, indicating that the light distribution inside the photobioreactor must be considered for the rational design and scale-up of photobioreactors. Therefore, a light distribution coefficient (Kiv), defined as the cell concentration at which 50% of the photobioreactor volume receives enough light for photosynthetic growth, was proposed. It was shown that linear growth rates increased with increase in Kiv but the data were scattered, and that at a constant Kiv, a linear relationship was observed between the linear growth rate and the Eₜ/V. Similarly, when the Eₜ/V was held constant, there was a good correlation between the Kiv and the linear growth rate. A light supply coefficient, defined as Eₜ/V.Kiv was then proposed as an index of the light supply efficiency of photobioreactors. Good correlation was found between the light supply coefficient and the linear growth rates of both C. pyrenoidosa and Spirulina plantensis in cuboidal photobioreactors of various sizes as well as in various types of both internally illuminated and externally illuminated cylindrical photobioreactors. (Ogbonna et al., 1995, Light supply coefficient: A new engineering parameter for photobioreactor design, J Ferm and Bioeng 80:369-376.)

The present light arrays allow the light supply coefficient to be at highly beneficial levels, e.g., due to the ability to provide photosynthetically effective illumination throughout the volume of culture medium encompassed by the light array.

The effectiveness of the present light array design is supported by a study on correlations between certain illumination parameters and growth rates for particular algal species. Initially it was shown that there is poor correlation between the incident light intensities, the average light intensities and the light energy supplied per unit photobioreactor volume (Eₜ/V and linear growth rates of Chlorella pyrenoidosa in cuboidal photobioreactors of various sizes. It was also found that at a given Eₜ/V, the linear growth rates decreased with increase in the photobioreactor depth, indicating that the light distribution inside the photobioreactor must be considered for the rational design and scale-up of photobioreactors. Therefore, a light distribution coefficient (Kiv), defined as the cell concentration at which 50% of the photobioreactor volume receives enough light for photosynthetic growth, was proposed. It was shown that linear growth rates increased with increase in Kiv but the data were scattered, and that at a constant Kiv, a linear relationship was observed between the linear growth rate and the Eₜ/V. Similarly, when the Eₜ/V was held constant, there was a good correlation between the Kiv and the linear growth rate. A light supply coefficient, defined as Eₜ/V.Kiv was then proposed as an index of the light supply efficiency of photobioreactors. Good correlation was found between the light supply coefficient and the linear growth rates of both C. pyrenoidosa and Spirulina plantensis in cuboidal photobioreactors of various sizes as well as in various types of both internally illuminated and externally illuminated cylindrical photobioreactors. (Ogbonna et al., 1995, Light supply coefficient: A new engineering parameter for photobioreactor design, J Ferm and Bioeng 80:369-376.)

The present light arrays allow the light supply coefficient to be at highly beneficial levels, e.g., due to the ability to provide photosynthetically effective illumination throughout the volume of culture medium encompassed by the light array.

Another way of considering the present invention is in the context of algal growth in top illuminated ponds. In general, for incident light that is initially of an appropriate intensity for growth, the intensity will attenuate rapidly as it passes through a cell suspension. The result is that growth effective light is only available in a thin layer. For example, in a pond with natural light, almost all of the growth occurs in the top approximately ½ inch, which can be referred to as a growth plane. The present light arrays essentially reproduce such growth planes within a tank, thereby eliminating the limitation of growth occurring only at the top surface and/or wall surface of the growth tank. This is accomplished by spacing light sources such that a plurality of growth planes is created. For example, with a rotating light array, two adjacent light rotation planes can be arranged spaced approximately two growth planes apart. That is, the spacing between the rotation planes is such that the lights traveling in one of the rotation planes provide effective illumination to a distance approximately equal to one growth plane, and the lights traveling in the adjacent rotation plane provide effective illumination a distance approximately equal to one growth plane. In this way, effective illumination is provided across the entire distance between light rotation planes.

The distance equal to one growth plane will depend on at least the cell density, the light requirements for the organism being cultured, and the initial light intensity from the light wand. In many cases, a light plane will be in the range of about 0.5 to 2.5 cm.

### Nutrient Dispersal

In addition to providing illumination, the light arrays can provide nutrient dispersal, e.g., CO2 and/or nitrogen. For example, the rotating axle can include a dispersal mechanism or tube to distribute nutrients such as CO₂ and nitrates.

Such dispersal mechanism can, for example, involve passage of such nutrients through or along a rotating array axle. The nutrients can be passed into the surrounding medium, e.g., through openings in the passageway and/or can be directed (e.g., using tubing) along at least some of the light wands and passed into the medium along or proximal to the wands. Of course, nutrients may also be injected or otherwise passed into the medium with other mechanisms and at different locations.

### Illumination and Nutrient Control

In a functional system, it is advantageous to control, actively and/or as part of the basic design of the system, illumination and/or provision of nutrients. Such control can be applied to a number of different parameters.

For example, the process optimization of the light system can be further enhanced by the timing of lights in an artificial setting. In natural settings, it has been observed that diurnal cycles are critical to growth of many photosynthetic algae, and relates to CO2 utilization and carbon fixation by the algae. As part of the cycles, CO2 is trapped during the night for release and utilization during the day. At dawn, when light becomes available, the rate of consumption of carbon dioxide through photosynthesis exceeds that of CO2 production through respiration and, as a result, the store of carbon dioxide is depleted and algal growth becomes limited. In other words, the carbon dioxide accumulated during the night hours is stored for use in the daytime hours. Carbon dioxide concentrations as high as 25 mg/L have been observed at night in lagoons. (Williford, H. K. and Middlebrooks, E. J. (1967). “Performance of field-scale facultative wastewater treatment lagoons.” J. WPCF, 39, 2008-2019.)

In effect the natural diurnal cycle is a natural metering cycle for daytime CO2 uptake and fixation. However, in a photobioreactor, by maintaining lights at the correct frequency and increasing photonic activity in close proximity to the bio-mass, we have found that CO2 fixation or uptake can be substantially extended, even as much as extending it such that CO2 fixation occurs on a twenty four hour basis, eliminating the diurnal cycle. In such tests, we have found that this storage/release cycle can be artificially extended to a 24 hour rotation as now control of CO2 release is timed by lighting which is synchronized to the uptake of carbon by the algae rather than to a diurnal cycle approximation.

In photosynthesis, the cycle of storage and release can be directly measured in ORP and pH. We find that when the bio-mass is absorbing CO2, it releases energy in the form of a negative hydroxyl (OH⁻). This brings the pH up and its counter indicator, the oxidation reduction potential (ORP) (units in mV), down. By carefully monitoring the pH and/or ORP levels, we can determine when the biomass is able to uptake CO2.

The use of light sources arranged such that the algae being cultured is in close proximity to the light sources, e.g., often within about a quarter inch to one inch, can be beneficially combined with selection and/or active control of the specific bandwidths of radiance (i.e., the light wavelengths) which are provided for the illumination. In general plant physiology, the term Photosynthetically Active Radiation (PAR) refers to the radiation in the range of wavelengths between about 400 nm and 720 nm. This is the energy that is absorbed by the assimilation pigments in blue-green algae, green algae and higher order plants. The wavelengths for the lower limit (400 nm) and upper limit (720 nm) are not entirely rigid. Photosynthetic reactions have, for example, been established in some algae at wavelengths shorter than 400 nm. In general, the lower limit depends on the structure and the thickness of the leaf as well as on the chlorophyll content. Some research projects have shown 700 nm as the upper wavelength limit.

For plant physiology, this range can be divided into three narrower bands:
400 nm to 510 nm: strong light absorption by chlorophyll, high morphogenetic effect
510 nm to 610 nm: weak light absorption by chlorophyll, no morphogenetic effect
610 nm to 720 nm: strong light absorption by chlorophyll, high morphogenetic and ontogenetic effect. (Tutorials section of Gigahertz Optics web site).

The limited bandwidths effective for photosynthesis can be used to increase the energy efficiency of a photobioreactor by reducing or excluding ineffective wavelengths and/or damaging (UV) wavelengths.

The light wavelengths absorbed by the light assimilation pigments of photosynthetic bacteria can vary from that indicated above for plants, in some cases including wavelengths longer and/or shorter than the plant PAR.

Still further, timing the exposure to light to coincide with the CO2 fixation pattern of the cells can further improve energy efficiency and/or enhance photosynthetic growth. Thus, for example, it appears that the algal cells do not require continuous light, but rather can be illuminated intermittently to imitate photosynthesis reaction, with the series of reactions running to completion during a relatively dark interval. It appears that an approximately 6 second cycle is effective, although other cycles or intervals may also be used. That is, within each 6 second cycle (or other interval), light is provided to a particular volume of culture medium for a short interval, e.g., about 0.1 to 1.0 second, followed by little or no light. This can be accomplished, for example, using the rotating light wands such that a light wand is in position to illuminate a particular spatial position on such cycle. For example, a flat array of 5 light wands rotating at 2 rpm will provide such 6 second cycle.

Thus, as described above, the present light arrays are effective for providing effective, or even optimized, light intensities to a large fraction or even substantially all of the culture medium encompassed by the light array over a broad range of cell concentrations. This is accomplished by placing light sources with appropriately selected and/or controlled light intensities such that the light needs to traverse only short paths (e.g., about 0.5 to 2.5 cm) in order to contact substantially all of the cells in the light array volume. CO2 uptake directly diminishes as a function of the distance of algae to light (assuming the maximum light intensity is not so great as to damage the algae). Therefore, by placing light sources (e.g., light wands) in the tank at narrow intervals, the algae passing between adjacent light sources can be exposed to photosynthetically effective light intensities. That is, the reduction in light intensity across the medium in the gap between adjacent light sources is kept relatively small, and therefore the reduction in CO2 uptake by algae across that gap is also kept relatively small. Therefore, with appropriately selected path lengths, a large fraction of the cells will be within a distance from at least one light source such that the cells will receive photosynthetically effective light intensity. Such configuration of light sources such that essentially all of the algae will be exposed to light that is of appropriate intensity for photosynthesis can significantly increase the total photosynthetic activity of the culture. Provision of light of proper wavelengths and/or provision of light on a beneficial illumination schedule can then enhance energy usage and/or growth.

### Tanks

As indicated above, the present light arrays can be advantageously used in any of a broad range of tank shapes and sizes. For any tank size and shape, it is beneficial for as much as possible, (preferably essentially all) of the culture medium in the tank to be exposed to light at close proximity to light sources of a light array. For example, for a cylindrical tank, a light array can be located and sized such that much of the volume in the tank will be within the volume described by rotation of the light array. Alternatively, the combination of rotation of the light array and flow of the bulk fluid results in substantially all of the tank fluid being exposed to effective illumination.

For larger tanks, especially tanks that are not cylindrical (e.g., rectangular tanks), it can be beneficial to utilize multiple light arrays, which may be the same or different. Use of multiple light arrays is beneficial, for example, to provide more consistent light exposure to the microorganisms being grown, to avoid dead spots in the tank, to prevent the difference between light exposure at the tips of light wands and the light exposure near the axle from being too great, and/or to prevent shear generated near the tips of light wands from being excessive due to speeds that are too high. For example, in a square tank, 5 light arrays may be used, a larger one centrally located and a small one in each of the four corners. In another example, in a rectangular tank that is elongated horizontally, a series of light arrays of equal size may be distributed along the length of the tank. Many other arrangements can also be selected based, e.g., on the particular tank size and shape and/or culture requirements of the organism to be grown.

Tanks can also be constructed of a variety of materials. For example, tanks may be constructed of plastics such as polycarbonate, or glass, or of metal such as stainless steel. The material and thickness can be selected based on normal considerations such as tank size, cleaning requirements, and effects on organisms to be grown, among others.

Tanks may also be made to enhance light usage by means of a reflector (e.g., a reflective layer or separate reflector) oriented to reflect light that would escape through the tank walls (and/or bottom and/or top) back into the culture medium. It is preferable that light emission and such recovery reflection are properly balanced to avoid photobleaching or other deleterious effects on organism growth and/or on production of desired product. Alternatively or in addition to light recovery, light can be transmitted into the culture medium from light emitters at the tank wall(s) or by transmission through tank walls.

### Exemplary Light Arrays, Illumination, and Organism Growth

A number of different exemplary features and options of the present invention are described below.

The use of light arrays as described for the present invention provides significant advantages due to the ability to control the illumination provided to the cultured microorganisms appropriately. As indicated above, these light arrays can be configured in a number of different ways. Advantageous light array configurations include rotating light arrays. Such configurations can enhance biomass growth by the close contact between the growing photosynthesizing organisms and light. At the same time, the invention can provide great scalability, something not reasonably achievable by many of the prior designs. Further, the light arrays, e.g., rotating light arrays, can be used in pre-existing tanks and/or sized according to desired spatial distributions and/or movement speeds and/or production rates. A further advantage is that the present systems can be constructed at relatively low cost.

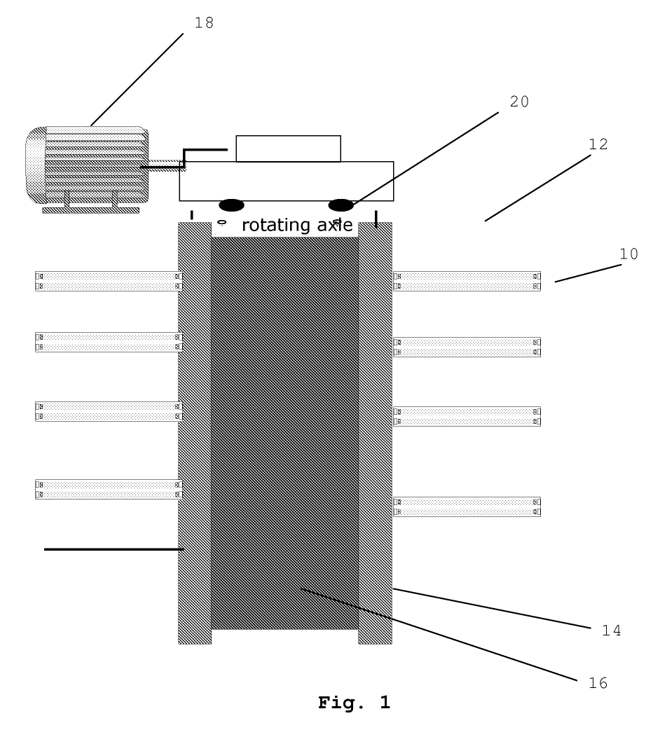

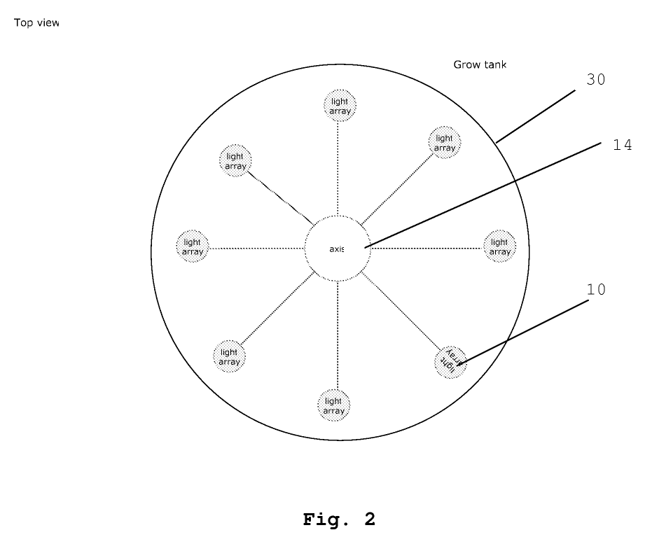

For example, a rotating light array can be, but is not necessarily, symmetrical about a central axis. A simplified illustration of such a light array is shown in FIG. 1 and FIG. 2. As shown in the cross-sectional side view diagram of FIG. 1, the light wands 10 of the light array 12 are mounted on a central shaft or axle 14 providing a central axis. The illustrated axle can have a passageway or duct 16 (which commonly is centrally located) which can be used, for example, to feed nutrients into the culture medium. The motor 18 drives the axle 14, e.g., through a slip ring configuration 20 so as to prevent tangling of wires, although direct drive can also be used. The motor is set to rotate at a desired speed or to follow a programmed speed profile over time, e.g., through pre-programming or variable speed drive. The light wands 10 can be placed at intervals along the axle so as to promote fluid flow and maximum effective illumination along the narrow gaps between the light wands.

The top view of the light array provided in FIG. 2 shows a view of the rotating axle within a tank 30. In this view, a single flat array of light wands 10 is visible. The shape of the tank can be of any type, e.g., circular, square, etc.

A light array similar to that shown in FIG. 1 and FIG. 2 was tested for growth of Nannochloropsis in a cylindrical growth tank to compare energy usage between the light array system using cold cathode fluorescent lights and a simple externally illuminated mechanically mixed tank using a grow light. The light array included flat arrays of five lights at 1.5 inch intervals along the central axle, and was rotated at one rpm. The test showed that the light array system was substantially more efficient in producing biomass, requiring much less energy per unit biomass increase.

### Systems and System Process Control

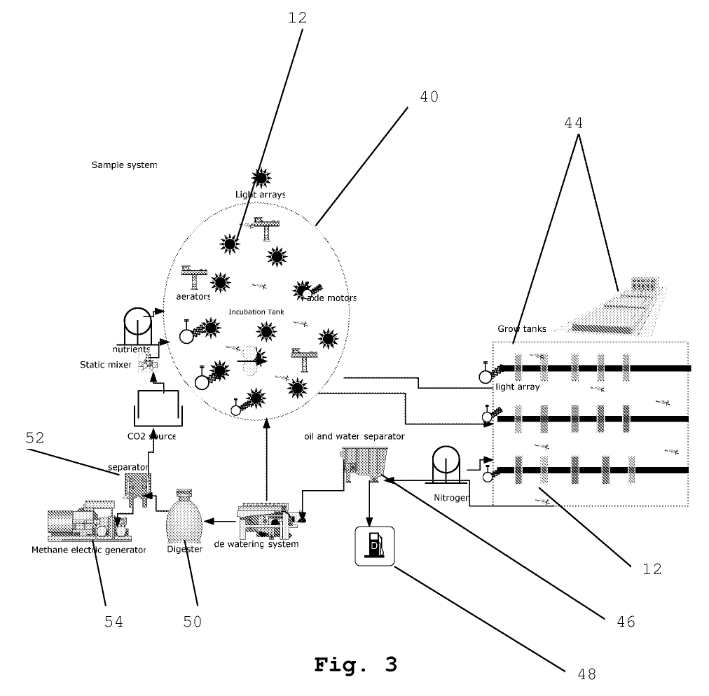

Process systems utilizing the present invention can be configured in a variety of ways. An exemplary process system utilizing a light array, e.g., as illustrated in FIG. 1 and FIG. 2, is shown in FIG. 3. The diagram shows an illustrative configuration of a system utilizing light arrays. This system includes a complete power generation scheme which includes algae as a CO2 capture system. The products of the system are fuels and electricity through co-generation of methane gas.

Efficiency is accomplished by using essentially all the elements of the biomass. It is understood that by configuring the type of algae stock (with the potential use of bacteria), one can achieve substantial amounts of fuels (e.g., biodiesel) and/or methane and/or overall biomass. Such method can be used without further modification, or can be use for production of methanol and/or other compounds in processes for which methane or methanol is a feed stock.

In this system the light arrays 12 are positioned directly in a tank 40, such as a sewage or sluice or aeration pond. The algae stock's growth is optimized to capture as much CO₂ as is possible. The biomass is then disgorged into what could be referred to as a product growth tank 44, where maximum lipid value is created in time, also utilizing light arrays 12.

The biomass is then fed through a separator 46; where constituent parts, such as sugars, oil and/or other valuable products are captured. In the illustrated system, lipids are separated for use in biodiesel production 48.

The biomass from which desired components have been separated is then fed to an anaerobic digester 50, where the biomass is then processed for methane gas. The gas is then stripped of its constituent CO₂, gas through a gas separator 52, e.g., a conventional bubbler. The CO₂ is re-introduced to the biomass growth tank 40 and/or the product growth tank 44. The methane is burned (optionally along oxygen captured from the growth tanks) for clean energy generation in a conventional methane burning generator 54.

The described process flow is only one of many potential uses of the present invention. The present light arrays bring to the culturing of algae and other photosynthesizing microorganisms the advantage of a rapid, controllable growth method. Further, the design can be configured in modular fashion, providing easy scale-up of capacity. Still further, the light arrays can be retrofit to existing systems and/or systems incorporating the light arrays can be made portable.

Thus, the light arrays and systems utilizing such arrays are applicable to any of a number of different processes, for example, for the creation of lipids for bio-fuels, remediation systems for flue stack cleaning or CO₂ fixation, waste treatment, and the like. Advantageously, these light array armatures can be adapted to creation of biomass inexpensively and with low capital costs.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the tank size, shape, and/or construction materials; to the light array size, number, placement, and/or light configuration; to the selection of cultured organism, to the culture media used, and/or to the product obtained from the cultured cells. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention. Further, specification of a numerical range including values greater than one includes specific description of each integer value within that range.

Thus, additional embodiments are within the scope of the invention and within the following claims.

What is claimed is:

1. A culture system for photosynthetic microorganisms, comprising
a culture tank;
a rotatable light array positioned within said tank, wherein said light array comprises an axle, a rotational drive connection linked to said axle, a plurality of light emitting projections extending outward from said axle and positioned such that essentially all culture media passing between adjacent light emitting projections will receive photosynthetically effective illumination; and
a rotational drive, wherein said drive is linked to and provides power through said rotation drive connection to rotate said rotatable light array.

2. The culture system of claim 1, wherein said rotatable light array comprises a plurality of flat arrays distributed along said axle.

3. The culture system of claim 2, wherein adjacent flat arrays are separated a distance of 0.5 to 5 cm.

4. The culture system of claim 2, wherein a flat array comprises at least 4 light wands.

5. The culture system of claim 1, wherein the wavelengths of light emitted from said light wands is selected to provide effective photosynthesis while reducing power consumption.

6. The culture system of claim 1, wherein during normal use, said rotatable light array rotates at 0.5 to 10 rpm.

7. The culture system of claim 1, further comprising at least one channel for distributing nutrients to culture media in said tank.

8. The culture system of claim 1, wherein said axle is positioned substantially vertically in said tank.

9. The culture system of claim 1, wherein said axle is positioned substantially horizontally in said tank.

10. The culture system of claim 1, comprising a plurality of said light arrays.

11. The culture system of claim 1, further comprising a light controller which controls at least one parameter selected from the group consisting of light intensity, illumination periods, and light wavelength for light emitted from said light wands.

12. The culture system of claim 11, further comprising at least one culture medium sensor, wherein said controller receives signals from said sensor and controls light emitted from said light wands at least in part as a function of said signals.

13. The culture system of claim 12, wherein said signals correspond to pH or oxidation reduction potential (ORP) or both.

14. A light distribution array, comprising
an axle;
a plurality of light emitting wands extending from and distributed along said axle, wherein said light emitting wands are spaced apart a distance of 0.5 to 5 cm along the axis of said axle.

15. The light distribution array of claim 14, comprising a plurality of flat arrays of light emitting wands.

16. The light distribution array of claim 14, wherein said light emitting wands are distributed along said axle over a distance of at least 0.5 m.

17. The light distribution array of claim 14, wherein said light emitting wands comprises at least one light source which is a light emitting diode (LED), a cold cathode fluorescent light (CCFL), or an external electrode fluorescent light (EEFL).

18. A system for carbon fixation and product recovery, comprising
a culture system comprising
a culture tank;
a rotatable light array positioned within said tank, wherein said light array comprises an axle, a plurality of light emitting projections extending outward from said axle and distributed along said axle, and positioned such that essentially all culture medium passing between adjacent light emitting projections will receive photosynthetically effective illumination, and a rotational drive connection; and
a rotational drive, wherein said drive is linked to and provides power through said rotation drive connection to rotate said rotatable light array;
a process controller which monitors at least one culture parameter indicative of photosynthesis and regulates at least one of light duration, light intensity, and light wavelength.

19. The system of claim 18, further comprising an oil extractor.

20. The system of claim 18, further comprising a biomass digester which receives biomass from said culture tank.

21. The system of claim 18, further comprising an electrical generator powered by at least one product of the system.

22. The system of claim 21, wherein said product is an oil-based fuel.

23. The system of claim 21, wherein said product is biomass.

24. The system of claim 21, wherein said product is methane.

25. The system of claim 20, further comprising a biomass digester, wherein CO2 produced in said digester is used as a nutrient in said culture tank.

26. A method for growing photosynthesizing microorganisms, comprising
exposing photosynthetic microorganisms in a growth medium in a photobioreactor to photosynthetically effective light from a light array, wherein said light array comprises a plurality of light wands spaced such that substantially all of the growth medium between successive light wands in said light array receive photosynthetically effective illumination.
